(19) United States
(12) Patent Application Publication
(10) Pub. No.: US 2006/0190072 A1
(43) Pub. Date: Aug. 24, 2006

(54) Flexible cells for axially interconnecting stent components

(76) Inventor: Gladwin S. Das, Arden Hills, MN (US)

Correspondence Address: KONDZELLA AND CYR, PONDVIEW PLAZA, SUITE 114, 5850 OPUS PARKWAY, MINNETONKA, MN, 55343, US

(21) Appl. No.: 11/341,351

(22) Filed: Jan. 27, 2006

Related U.S. Application Data
(63) Continuation-in-part of application No. 11/045,927, filed on Jan. 28, 2005.

(51) Int. Cl. A61F 2/06

(52) U.S. Cl. 623/1.15

(57) ABSTRACT

Interconnects for connecting axially expandable segments of stents are disclosed. The interconnects include a proximal connector, a first arm, a second arm and a distal connector. The connectors secure the interconnect to the adjacent axially expandable members. The first arm and the second arm provide flexible elements of the interconnect to confer a degree of axial flexibility between the segments.

### CROSS-REFERENCE TO RELATED APPLICATIONS

The present application is a Continuation-In-Part Application of co-pending U.S. patent application Ser. No. 11/045,927, filed Jan. 28, 2005, entitled FLEXIBLE CELLS FOR AXIALLY INTERCONNECTING STENT COMPONENTS.

### BACKGROUND OF THE INVENTION

1. Field of the Invention

The present invention relates to medical stents and, more particularly, to methods and apparatus which interconnect expandable units within a stent.

2. Background of the Related Art

In recent years a corrective procedure, percutaneous transluminal coronary angioplasty, and devices known as balloon angioplasty catheters have been widely used to correct stenotic conditions within arteries, particularly coronary arteries, in a relatively efficient manner. An angioplasty procedure generally includes inserting a deflated balloon, mounted on a catheter, within the affected vessel or artery at the point of a stenosis. The balloon is then inflated to physically force the dilation of the partially occluded vessel.

Unfortunately, a substantial percentage of patients who have had balloon angioplasty redevelop the stenosis in a relatively short period of time. The reoccurrence of stenosis, termed restenosis, typically becomes evident within 6 months of the angioplasty procedure and may affect 30 to 40 percent of patients. The percentage of patients who have reoccurring stenoses following angioplasty is generally reduced by installing a “scaffolding” device, known as a stent, at the site of the stenosis.

Stents are generally tubular devices, frequently made of a thin-walled metallic or woven material. Usually, a pattern of apertures, openings or holes is defined around the circumference of the stent along most of the length of the stent. A stent is guided to the stenosis by catheter and expanded to expand the lumen wall and provide support to the lumen wall so as to keep the lumen substantially open. While coronary and other arterial stenoses are common applications for stenting, stents can also be used to treat narrowings in any hollow or tubular organ or body lumen, such as the esophagus, urethra, biliary tract, and the like.

Stents may be constructed from a variety of materials, such as stainless steel, Elgiloy, Nitinol, shape memory polymers, and the like. They may be formed by a variety of methods. For example, a stent may be formed by etching or cutting the stent pattern from a tube or section of stent material; or a sheet of stent material may be cut or etched according to a desired stent pattern, whereupon the sheet may be rolled or otherwise formed into the desired tubular or bifurcated tubular shape of the stent; or one or more wires or ribbons of stent material may be braided or otherwise formed into the desired shape and pattern.

Stents are typically provided in two fundamental configurations termed self-expanding stents and balloon expandable stents. Combinations or hybrids of these two fundamental configurations have also been developed that have some characteristics of both self-expandable and balloon expandable stents. Self-expanding stents are generally spring-like devices that are inserted in the body passageway in a contracted state within a delivery catheter or introducer. A self-expanding stent is biased so as to expand upon release from the delivery catheter. When released, the self-expanding stent reconfigures from a contracted to an expanded state. The self-expanding stent tends to increase to a final diameter dependent on the size and configuration of the stent and the elasticity of the body passageway.

In contrast, a balloon expandable stent requires assistance from a balloon to expand into position. A balloon expandable stent is mounted over a balloon attached to the distal end of a catheter. The balloon expandable stent is guided by the catheter to the proper position at the stenosis. Then, the balloon is inflated to expand the stent radially outward into position. The amount of force applied is at least that necessary to maintain the patency of the body passageway. Once the stent is properly expanded, the balloon is deflated and withdrawn from the patient.

Stents need to be axially flexible for tracking through tortuous lumen of the human body. In order to make a stent axially flexible, a stent may be made in segments where the segments are connected together by elastic interconnects. The use of interconnects for connecting various segments of the stent has, to some extent, satisfied the need for axial flexibility. However, existing interconnects have certain limitations based upon the mechanisms by which a stent confers a physiological benefit.

The underlying mechanism for the physiological benefit produced by a stent may be as simple as preventing immediate elastic recoil of the luminal wall and maintaining a large luminal cross-section for a few days after angioplasty. Continuous support by the stent along the luminal wall may be important. In addition, stent surfaces are frequently coated with various therapeutic compounds that prevent restenosis or have other beneficial effects. However, the surface area between stent segments in stents incorporating interconnects is relatively small and the resulting gaps between stent segments may become sites of restenosis perhaps due to the decreased support of the lumen by the stent over the gaps between stent segments or due to the decrease in surface area having a therapeutic coating biased against the lumen over the gaps between stent segments.

It would, therefore, be a significant advance in the art to provide interconnects that will enable the stent to navigate through tortuous bodily lumen and to conform to tortuous bodily lumen when expanded while providing sufficient surface areas to prevent gaps between stent segments.

### SUMMARY OF THE INVENTION

Apparatus and methods in accordance with the present invention may resolve many of the needs and shortcomings discussed above and will provide additional improvements and advantages as will be recognized by those skilled in the art upon review of the present disclosure.

The present invention provides a stent composed of radially expandable segments, where the radially expandable segments are connected by flexible interconnects. In various embodiments, the expandable stent of the present invention may be self-expandable upon deployment, may be expanded by enlarging an expandable balloon positioned within the stent, or may be of the hybrid type. The stent according to the present invention can be described based on a cylindrical coordinate system where the stent defines a longitudinal axis passing along the length of the stent and a radial axis normal to the longitudinal axis.

Embodiments of a stent according to the present invention include a plurality of radially expandable segments interconnected by a series of axially flexible interconnects. The radially expandable segments may be configured to support or otherwise contact the walls of a body lumen. The radially expandable segments may be configured from a single strand extending radially around the longitudinal axis of the radially expandable segment or may be formed in a wide variety of alternative radially expandable configurations. The radially expandable segments may generally expand so as to be symmetric in a radial plane. In other variations, the radially expandable segments may be unsymmetric or of biased symmetry in the radial plane. A radially expandable segment may have a constant cross-section along the axis of the stent or a variable cross-section along the axis of the stent and there may be variations between the different segments that compose the stent.

Adjacent radially expandable segments are connected by a plurality of flexible interconnects. These flexible interconnects are primarily configured to flex or compress in the axial direction parallel to the axis of the stent. The interconnects do not expand in the curvilinear plane defined by the circumference of the stent upon expansion of the stent. Rather, the interconnects expand or contract axially so as to allow articulation of the expandable segments of the stent so as to allow the stent to navigate through a curved lumen or to allow the stent to be deployed within a curved lumen. Upon expansion of the stent, the interconnects are designed to provide additional support to the body lumen and also to provide additional surface area for the elution of therapeutic agents.

The interconnects are placed around the circumference of a distal radially expandable segment and the circumference of a proximal radially expandable segment so as to link the distal and proximal radially expandable segments. The interconnects generally include a first arm and a second arm designed to flex so as to allow axial expansion or axial compression as the radially expandable segments articulate in response to a curved lumen.

The first arm and the second arm may be symmetric or may be differentially configured as required to confer desired flexural characteristics. The first arm and the second arm are secured between a proximal connector and a distal connector. The proximal connector is secured to the proximal end of the first arm and the second arm and to a proximal radially expandable segment so as to communicate compressive or expansive forces between the first arm, the second arm, and the proximal radial expandable segment. The distal connector is secured to the distal end of the first arm and the second arm and to a distal radially expandable segment so as to communicate compressive or expansive forces between the first arm, the second arm, and the distal radial expandable segment.

Typical designs for interconnects according to the present invention include various curved as well as angular configurations of the first arm and the second arm that may be expandable and compressible in the axial direction but not in the radial direction.

Stents according to the present invention feature an absence of potential tissue snagging structures. The expandable segments are able to articulate with respect to one another, which enables the stent to pass through otherwise tortuous passageways. The stents of the present invention are efficiently and easily produced using laser etching or chemical etching techniques and are amenable to good quality control at a relatively low cost. Other features and advantages of the invention will become apparent from the following detailed description, from the figures, and from the claims.

### BRIEF DESCRIPTION OF THE DRAWINGS

FIG. 1 illustrates a perspective view of an exemplary embodiment of an expandable stent in accordance with the present inventions;

FIG. 2 illustrates a side view of an exemplary embodiment of a stent in accordance with the present inventions in an unexpanded configuration positioned over a balloon of a balloon catheter within a bodily lumen of a patient which is shown in cross-section;

FIG. 3 illustrates a side view of an exemplary embodiment of a stent in accordance with the present inventions in an at least partially expanded configuration positioned over a balloon of a balloon catheter within a bodily lumen of a patient which is shown in cross-section;

FIG. 4 illustrates a plan view of an embodiment of a stent in accordance with the present inventions, showing the stent in a relaxed planar configuration;

FIG. 5 illustrates a plan view of an embodiment of a stent in accordance with the present inventions, showing the stent in a planar configuration bent to illustrate the expansion and compression of the interconnects;

FIG. 6 illustrates an enlarged plan view of an exemplary embodiment for an interconnect in accordance with the present inventions;

FIG. 7 illustrates an enlarged plan view of another exemplary embodiment for an interconnect in accordance with the present inventions;

FIG. 8 illustrates an enlarged plan view of another exemplary embodiment for an interconnect in accordance with the present inventions;

FIG. 9 illustrates an enlarged plan view of another exemplary embodiment for an interconnect in accordance with the present inventions;

FIGS. 10A, 10B, and 10C illustrate an enlarge plan view of an exemplary embodiment for an interconnect in accordance with the present inventions in a relaxed, a compressed and an extended position, respectively;

FIGS. 11A, 11B, and 11C illustrate an enlarge plan view of another exemplary embodiment for an interconnect in accordance with the present inventions in a relaxed, a compressed and an extended position, respectively;

FIGS. 12A, 12B, and 12C illustrate an enlarge plan view of another exemplary embodiment for an interconnect in accordance with the present inventions in a relaxed, a compressed and an extended position, respectively;

FIGS. 13A, 13B, and 13C illustrate an enlarge plan view of another exemplary embodiment for an interconnect in accordance with the present inventions in a relaxed, a compressed and an extended position, respectively;

FIGS. 14A, 14B, and 14C illustrate an enlarge plan view of another exemplary embodiment for an interconnect in accordance with the present inventions in a relaxed, a compressed and an extended position, respectively; and

FIGS. 15A, 15B, and 15C illustrate an enlarge plan view of another exemplary embodiment for an interconnect in accordance with the present inventions in a relaxed, a compressed and an extended position, respectively.

All Figures are illustrated for ease of explanation of the basic teachings of the present invention only; the extensions of the Figures with respect to number, position, relationship and dimensions of the parts to form the preferred embodiment will be explained or will be within the skill of the art after the following description has been read and understood. Further, the exact dimensions and dimensional proportions to conform to specific force, weight, strength, and similar requirements for various applications will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with an apparatus in accordance with the present invention.

### DETAILED DESCRIPTION OF THE INVENTION

The figures generally illustrate embodiments of a stent 10 including aspects of the present inventions. The particular exemplary embodiments of the stent 10 illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, many variations from the illustrated embodiments may be encompassed by the appended claims.

The present inventions provide stents 10 and associated methods. In accordance with the present inventions, a stent 10 will include two or more radially expandable segments 12 interconnected by an axially flexible interconnect 14. Stents 10 in accordance with the present inventions may be positioned and expanded within a lumen of a patient. Stents 20 in accordance with the present inventions may provide enhanced flexibility and increased surface area for purposes of drug elution and/or support of a lumen wall. In one aspect, stents 10 in accordance with the present inventions may be radially expanded with a balloon 16.

As generally illustrated throughout the Figures, stents 10 generally include two or more radially expandable segments 12 interconnected by an interconnect 14. The interconnect 14 is typically configured primarily for axial expansion and/or compression along the longitudinal axis 300 of stent 10. The stent 10 generally defines a longitudinal axis 300 along the length of the stent 10. The stent 10 further includes a proximal end 110 and a distal end 210 which, in the illustrated embodiments, are defined primarily for purposes of description. Other stents 10 incorporating aspect of the present inventions may include a proximal end 110 and a distal end 210 that are functionally distinct without departing from the scope of the present inventions. For exemplary purposes, stent 10 has been illustrated as a balloon expandable stent 10 including a balloon 16 extending through a lumen 18 defined by the stent 10. In another aspect, the stent 10 may be configured as a self expanding stent 10 or a hybrid of self expanding and balloon expandable stent 10 as will be recognized by those skilled in the art. The radially expandable segments 12 are configured to radially expand after insertion into a lumen.

The radially expandable segments 12 may be configured to support or otherwise contact the walls of a bodily lumen of a patient. The radially expandable segments 12 may be configured from a single strand 20 extending radially around the longitudinal axis 300 at a desired distance as is generally illustrated in the figures for exemplary purposes or may be formed in a wide variety of alternative radially expandable configurations as will be recognized by those skilled in the art. The strand 20 is generally illustrated with a linear portion 22 which extends parallel to and along the longitudinal axis 300 of the stent 10. A proximal loop 24 turns the strand 20 distally along the longitudinal axis 300 at a proximal end of the radially expandable segment 12. A distal loop 26 turns the strand 20 proximally along the longitudinal axis 300 at the distal end of the radially expandable segment 12. The radially expanding units 12 have been illustrated as generally expanding within a direction perpendicular to the longitudinal axis 300 of stent 10 for exemplary purposes. Upon review of the present disclosure, those skilled in the art will recognize variations of the expandable unit that may expand radially at an angle which is not perpendicular to the longitudinal axis 300.

Adjacent radially expandable segments 12 are connected to one another by interconnects 14. The interconnects 14 are configured to be axially expandable along the longitudinal axis 300 of stent 10. In one aspect, the interconnects 14 may be configured to be axially compressible along the longitudinal axis 300 of stent 10. The radially expandable segments 12 within the stent 10 may also contact or be biased against the walls of a bodily lumen to support or otherwise contact the bodily lumen. As illustrated for exemplary purposes, the interconnects 14 may be symmetrically positioned about the longitudinal axis 300 of the stent 10. In other aspects, the interconnects 14 may be asymmetrically positioned about the longitudinal axis 300 to provide the desired flex characteristics or other characteristics to a stent 10.

The interconnects 14 generally include a first arm 34 and a second arm 36 secured between a proximal connector 44 and a distal connector 46. The first arm 34 and the second arm 36 are generally configured to flex for purposes of axial expansion and/or compression of the interconnect 14. In one aspect, the axial expansion and/or compression of the interconnects 14 may permit the bending of the stent 10 along the longitudinal axis 300 such that at least a portion of the longitudinal axis 300 is curvilinear. The proximal connector 44 is secured to the proximal ends of the first arm 34 and second arm 36 to communicate compressive or expansive forces between the first arm 34, second arm 36 and the proximal radial expandable unit 12. The proximal connector 44 may be integrally formed with, welded to, adhesively bonded to or otherwise secured to the proximal ends of the first arm 34 and second arm 36 as will be recognized by those skilled in the art upon review of the present disclosure. The proximal connector 44 may extend linearly, when in a relaxed state, for a distance between the proximal end of the first arm 34 and second arm 36 and the point of connection to the proximal radially expanding segment 12. The distal connector 46 is secured to the distal ends of the first arm 34 and second arm 36 to communicate compressive or expansive forces between the first arm 34, second arm 36 and the distal radial expandable unit 12. The distal connector 46 may be integrally formed with, welded to, adhesively bonded to or otherwise secured to the distal ends of the first arm 34 and second arm 36 as will be recognized by those skilled in the art upon review of the present disclosure. The distal connector 46 may extend linearly, when in a relaxed state, for a distance between the distal ends of the first arm 34 and second arm 36 and the point of connection to the distal radially expanding segment 12.

In one aspect, the first arm 34 and the second arm 36 may be symmetrical about a central axis 302 extending between the proximal connector 44 and the distal connector 46. In another aspect, the first arm 34 and second arm 36 may be differentially configured, such as by size shape or materials, to confer desired flex characteristics to the stent 10. The proximal connector 44 connects the interconnect 14 to a proximally positioned radially expandable segment 12. The proximal connector 44 may be integrally formed with, welded to, adhesively bonded to or otherwise secured to a proximally positioned radially expandable segment 12 as will be recognized by those skilled in the art upon review of the present disclosure. The distal connector 46 connects the interconnect 14 to a distally positioned radially expandable segment 12. The distal connector 46 may be integrally formed with, welded to, adhesively bonded to or otherwise secured to a distally positioned radially expandable segment 12 as will be recognized by those skilled in the art upon review of the present disclosure. For purposes of the present disclosure, the radially expandable segment 12 positioned proximal to an interconnect 14 along the longitudinal axis 300 may be referred to as a proximal radially expandable segment 12. Further, the radially expandable segment 12 positioned distal to an interconnect 14 along the longitudinal axis 300 may be referred to as a distal radially expanding unit 12 for purposes of claiming the present inventions for purposes of the present disclosure.

FIG. 1 particularly illustrates an exemplary embodiment of a stent 10 in accordance with the present inventions. As illustrated, stent 10 includes four radially expandable segments 12 interconnected by a plurality of interconnects 14 symmetrically distributed about a longitudinal axis 300 for exemplary purposes. Each radially expandable segment 12 is configured from a strand 20 extending radially around the longitudinal axis 300. For exemplary purposes, the strand 20 is illustrated as substantially equidistant from the longitudinal axis 300 over the length of strand 20. The strand 20 is shown with a linear portion 22 extending parallel to the longitudinal axis 300 of the stent 10. A proximal loop 24 turns the strand 20 distally along the longitudinal axis 300 at a proximal end of the radially expandable segment 12. A distal loop 26 turns the strand 20 proximally along the longitudinal axis 300 at the distal end of the radially expandable segment 12. The interconnects 14 are shown attached to the radially expandable segments 12 at the proximal loops 24 of the distal expandable units 12 and at the distal loops 26 of the proximal expandable units 12 for exemplary purposes.

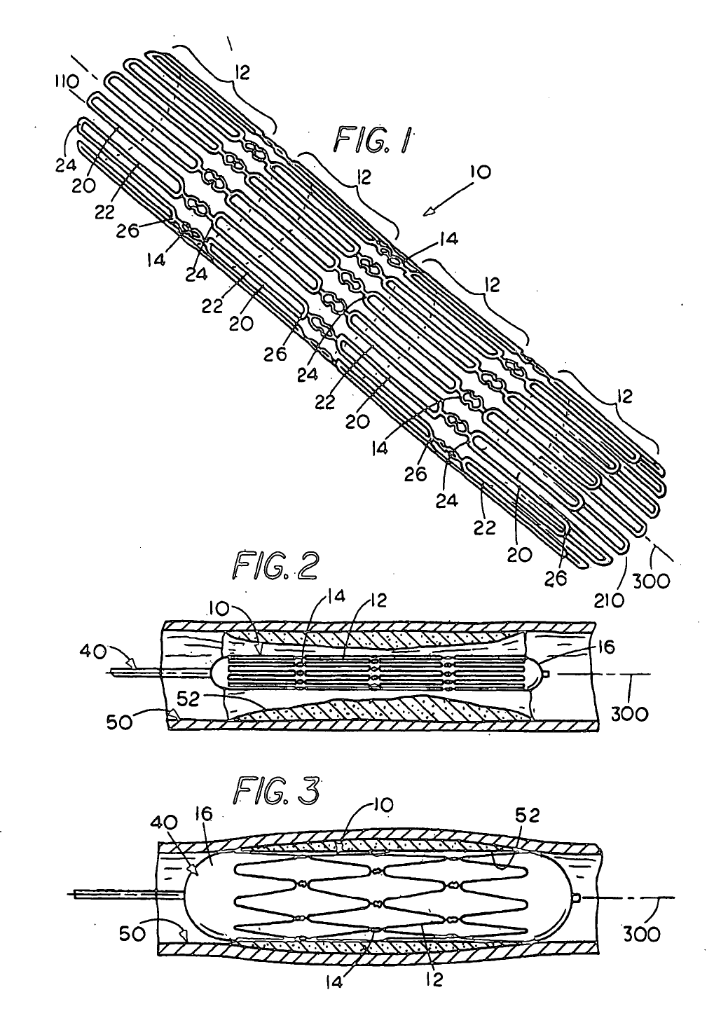

FIGS. 2 and 3 illustrate an exemplary embodiment of a stent 10 in accordance with the present inventions in a substantially un-expanded and at least partially expanded position, respectively. The stent 10 is illustrated as fitted over a balloon 16 of a balloon catheter 40. The stent 10 is also shown generally positioned within a portion of an artery 50 which is partially occluded by a stenosis 52. As illustrated in FIG. 3, once the stent 10 is appropriately located in the lumen of the artery 50, preferably spanning the stenosis 52, the radially expandable segments 12 of stent 10 can be expanded radially outward by inflating the balloon 16 of the balloon catheter 40. As balloon 16 expands, the stent 10 is brought into contact with and may alter the shape of the stenosis 52. After the radially expandable segments 12 of the stent 10 are fully expanded, the balloon 16 may be deflated and the balloon catheter 40 removed from the patient. Typically, with the expanded stent 10 positioned within the patient, the patency may be at least partially restored in the artery 50.

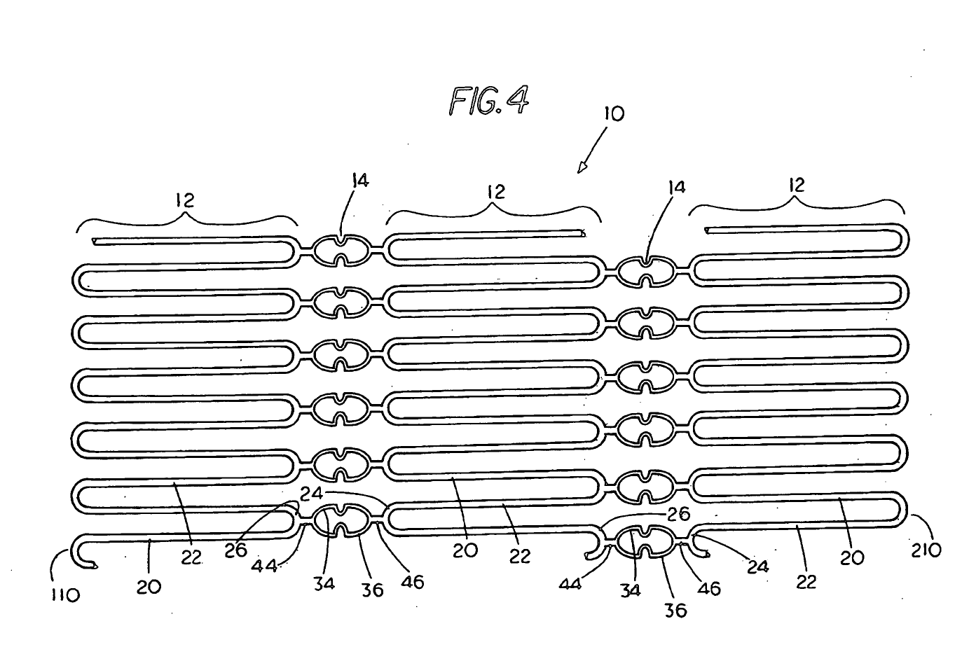

FIGS. 4 and 5 show plan views of an exemplary embodiment of a stent 10 in accordance with the present inventions in a planar configuration for purposes of illustration. FIG. 4 illustrates a stent 10 in accordance with the present inventions, showing the stent 10 in a relaxed configuration. FIG. 5 illustrates a stent 10 in accordance with the present inventions showing the stent 10 in a configuration where the stent 10 is bent along the longitudinal axis 300 to illustrate the expansion and compression of the interconnects 14 positioned about the periphery of the stent 10. Radially expandable segments 12 are shown connected by interconnects 14. Each of the interconnects 14 includes a proximal connector 34 which is secured to a proximal radially expandable segment 12 and a distal connector 36 which is secure to a distal radially expandable segment 12. A first arm 34 and a second arm 36 are secured between the proximal connector 34 and the distal connector 36. Interconnects 14 are configured to expand or contract in the axial direction, but not in the radial direction. FIG. 5 particularly illustrates the varying expansion of interconnects 14 about the periphery of a stent 10 as the stent 10 is flexed along its longitudinal axis 300. For illustrative purposes, the interconnects 14 have been labeled 14a through 14e in order of a substantially fully extended position to substantially relaxed position. Upon review of the present disclosure, those skilled in the art will recognize the implications on the expansion of the interconnects 14 upon the circularization of the planar illustrated embodiment of FIGS. 4 and 5.

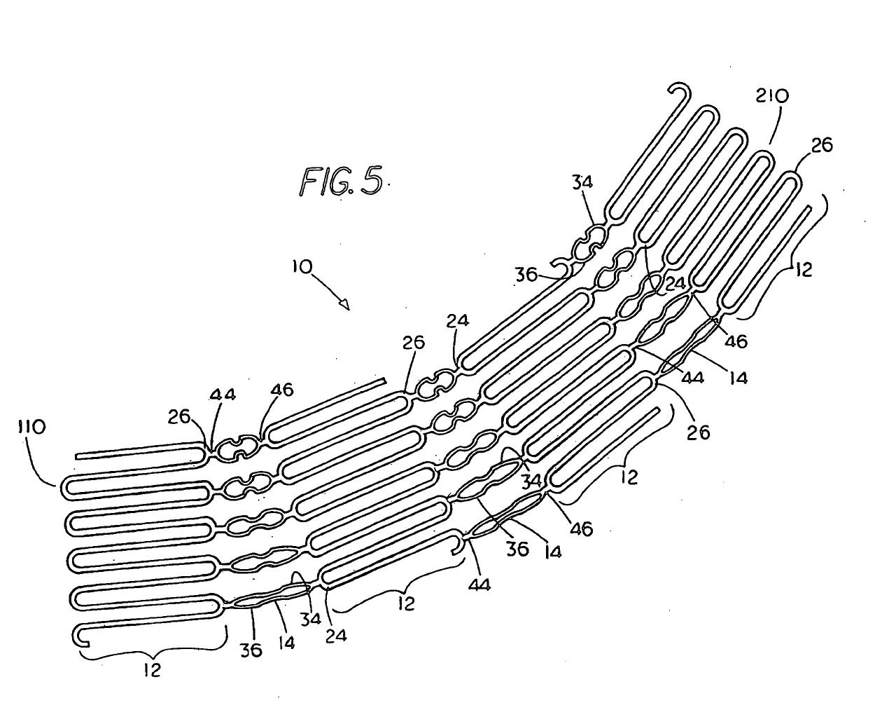

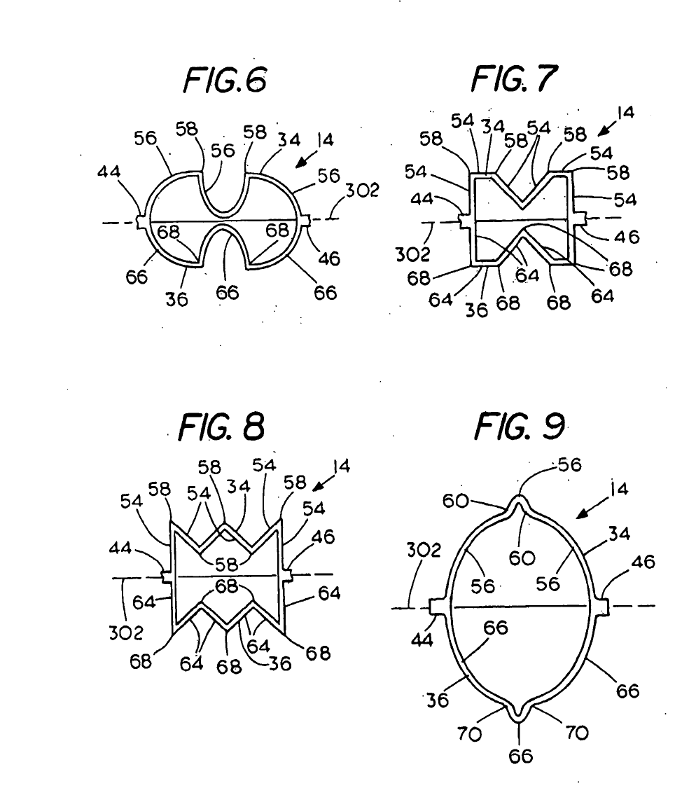

FIGS. 6 to 9 illustrate exemplary embodiments for interconnects 14 in accordance with the present inventions. The illustrated interconnects 14 include a proximal connector 44, a first arm 34, a second arm 36 and a distal connector 46. The first arm 34 and the second arm 36 are configured to enhance the flexibility of the stent 10 along the longitudinal axis 300 of the stent 10. In one aspect, the enhanced flexibility in accordance with the present inventions may permit the flexing of the stent 10 along its longitudinal axis without the deformation of the lumen defined by the stent 10. As illustrated, a central axis 302 may extend between the proximal connector 44 and the distal connector 46. Central axis 302 is typically substantially parallel to longitudinal axis 300. At least a portion of a linear distance of the proximal connector 44 and the distal connector 46 are illustrated extending along the central axis 302 for exemplary purposes. The first arm 34 and the second arm 36 in the illustrated embodiments are substantially symmetrical to one another about the central axis 302. The first arm 34 and the second arm 36 may lie substantially within a curved plane defined by the outer surface of the radially expandable segments 12. The first arm 34 may include one or more linear sections 54, curved sections 56 and angled transitions 58 to define a flexing region along at least a portion of the first arm 34. The linear sections 54, curved sections 56 angle transitions 58 and curved transitions 60 generally extend from the central axis 302 within the plane defined by the outer surface of the radially expandable segments 12. The second arm 36 may include one or more linear sections 64, curved sections 66 and angled transitions 68 to define a flexing region along at least a portion of the second arm 36. The linear sections 64, curved sections 66, angle transitions 68 and curved transitions 70 generally extend from the central axis 302 in the opposite direction of the linear sections 54, curved sections 56, angled transitions 58 and curved transitions 60 of the first arm 34. The linear sections 64, curved sections 66, angle transitions 68 and curved transitions 70 generally lie within the plane defined by the outer surface of the radially expandable segments 12. The curved portions are typically defined as concave or convex relative to the central axis 302.

As particularly illustrated in FIG. 6 for exemplary purposes, the first arm 34 extends distally from a proximal end secured to the proximal connector 44 and defines a convex curved section 56 up to a first angled transition 58 followed by a concave curved section 56 up to a second angled transition 58 followed by a second convex curved section 56 and terminating at the distal connector 46. The second arm 36 is illustrated as substantially symmetrical about the central axis 302 to the first arm 34 for exemplary purposes. Particularly, the second arm 36 extends distally from a proximal end secured to the proximal connector 44 and defines a convex curved section 66 up to a first angled transition 68 followed by a concave curved section 66 up to a second angled transition 68 followed by a second convex curved section 66 and terminating at the distal connector 46 and terminating at the distal connector 46.

As particularly illustrated in FIG. 7 for exemplary purposes, the first arm 34 extends distally from a proximal end secured to the proximal connector 44 and defines a first linear section 54 extending perpendicular from the central axis 302 up to a first angled transition 58 followed by a second linear section extending parallel to the central axis 302 up to a second angle transition 58 followed by a third linear section 54 extending toward the central axis 302 followed by a third angled transition 58 followed by a fourth linear section 54 extending away from the central axis 302 up to a fourth angled transition 58 followed by a fifth linear section 54 extending parallel to the central axis 302 up to a fifth angled transition 58 followed by a sixth linear section 54 extending perpendicular to the central axis 302 and terminating at the distal connector 46. The second arm 36 is illustrated as substantially symmetrical about the central axis 302 to the first arm 34 for exemplary purposes. Particularly, the second arm 36 extends distally from a proximal end secured to the proximal connector 44 and defines a first linear section 64 extending perpendicular from the central axis 302 up to a first angled transition 68 followed by a second linear section extending parallel to the central axis 302 up to a second angle transition 68 followed by a third linear section 64 extending toward the central axis 302 followed by a third angled transition 68 followed by a fourth linear section 6 extending away from the central axis 302 up to a fourth angled transition 68 followed by a fifth linear section 64 extending parallel to the central axis 302 up to a fifth angled transition 68 followed by a sixth linear section 64 extending perpendicular to the central axis 302 and terminating at the distal connector 46.

As particularly illustrated in FIG. 8 for exemplary purposes, the first arm 34 extends distally from a proximal end secured to the proximal connector 44 and defines a first linear section 54 extending perpendicular from the central axis 302 up to a first angled transition 58 followed by a second linear section extending toward the central axis 302 up to a second angle transition 58 followed by a third linear section 54 extending away from the central axis 302 followed by a third angled transition 58 followed by a fourth linear section 54 extending toward from the central axis 302 up to a fourth angled transition 58 followed by a fifth linear section extending away from the central axis 302 up to a fifth angled transition 58 followed by a sixth linear section extending perpendicular to the central axis 302 and terminating at the distal connector 46. The second arm 36 is illustrated as substantially symmetrical about the central axis 302 to the first arm 34 for exemplary purposes. Particularly, the second arm 36 extents distally from a proximal end secured to the proximal connector 44 and defines a first linear section 64 extending perpendicular from the central axis 302 up to a first angled transition 68 followed by a second linear section extending toward the central axis 302 up to a second angle transition 68 followed by a third linear section 64 extending away from the central axis 302 followed by a third angled transition 68 followed by a fourth linear section 64 extending toward from the central axis 302 up to a fourth angled transition 68 followed by a fifth linear section extending away from the central axis 302 up to a fifth angled transition 68 followed by a sixth linear section extending perpendicular to the central axis 302 and terminating at the distal connector 46.

As particularly illustrated in FIG. 9 for exemplary purposes, the first arm 34 extends distally from a proximal end secured to the proximal connector 44 and defines a convex curved section 56 up to a first curved transition 60 followed by a second convex curved section 56 up to a second curved transition 60 followed by a second convex curved section 56 and terminating at the distal connector 46. The second arm 36 is illustrated as substantially symmetrical about the central axis 302 to the first arm 34 for exemplary purposes. Particularly, the second arm 36 extends distally from a proximal end secured to the proximal connector 44 and defines a convex curved section 66 up to a first curved transition 70 followed by a concave curved section 66 up to a second curved transition 70 followed by a second convex curved section 66 and terminating at the distal connector 46 and terminating at the distal connector 46.

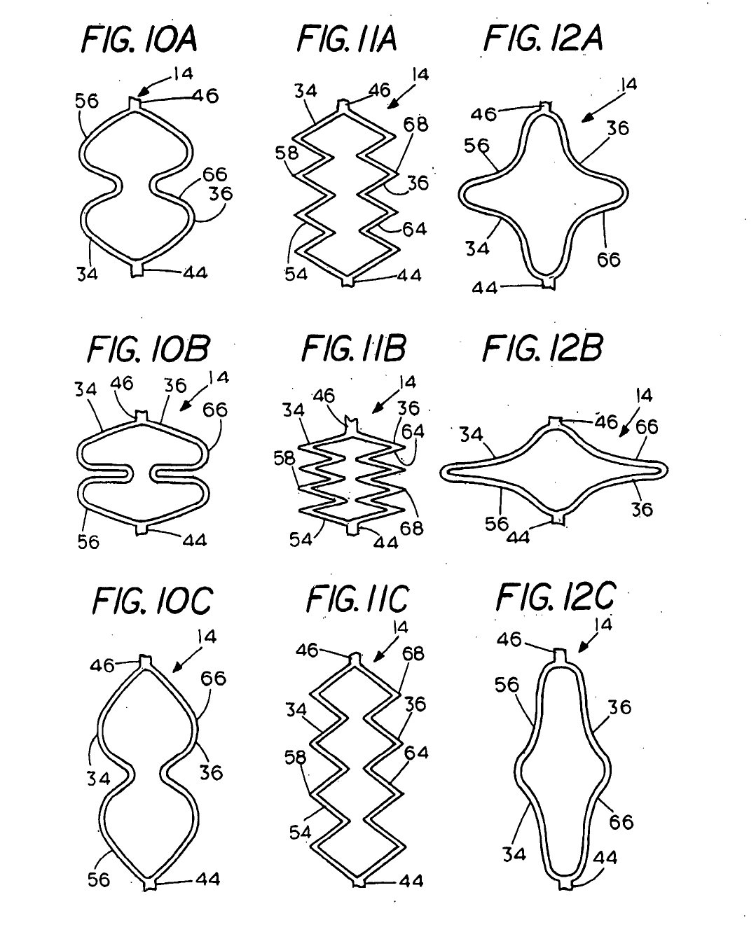

FIGS. 10A to 15C illustrate additional variations for symmetrical configurations of interconnects 14 in accordance with the present inventions. Each variation is illustrated in relaxed, at least partially compressed, and at least partially extended configurations for exemplary purposes.

FIGS. 10A to 10C illustrate an exemplary interconnect 14 having curved sections 56, 66 defining a first flexible lobe and a second flexible lobe axially along the interconnect 14. FIG. 10A illustrates the exemplary interconnect 14 in a substantially relaxed position. FIG. 10B illustrates the exemplary interconnect 14 in an at least partially compressed position. FIG. 10C illustrates the exemplary interconnect 14 in an at least partially expanded position.

FIGS. 11A to 11C illustrate another exemplary interconnect 14 including linear sections 54, 64 and angled transitions 58, 68 defining a saw tooth pattern axially along the interconnect 14. FIG. 11A illustrates the exemplary interconnect 14 in a substantially relaxed position. FIG. 11B illustrates the exemplary interconnect 14 in an at least partially compressed position. FIG. 11C illustrates the exemplary interconnect 14 in an at least partially expanded position.

FIGS. 12A to 12C illustrate another exemplary interconnect 14 curved sections 56, 66 defining a single flexible lobe. FIG. 12A illustrates the exemplary interconnect 14 in a substantially relaxed position. FIG. 12B illustrates the exemplary interconnect 14 in an at least partially compressed position. FIG. 12C illustrates the exemplary interconnect 14 in an at least partially expanded position.

FIGS. 13A to 13C illustrate another exemplary interconnect 14 having curved sections 56, 66 and curved transitions 60, 70 defining a first flexible lobe and a second flexible lobe axially along the interconnect 14 in an alternative configuration to the lobes illustrated in FIGS. 10A to 10C. FIG. 13A illustrates the exemplary interconnect 14 in a substantially relaxed position. FIG. 13B illustrates the exemplary interconnect 14 in an at least partially compressed position. FIG. 13C illustrates the exemplary interconnect 14 in an at least partially expanded position.

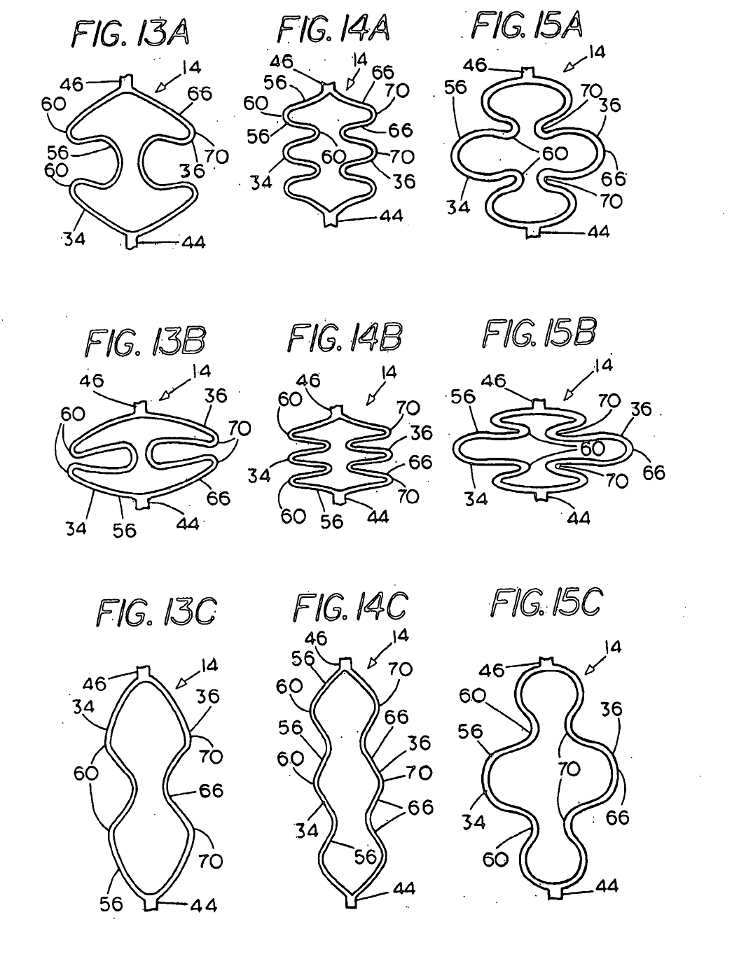

FIGS. 14A to 14C illustrate another exemplary interconnect 14 having curved sections 56, 66 and angled transitions 60, 70 defining a first flexible lobe, a second flexible lobe and a third flexible lobe axially along the interconnect 14. FIG. 14A illustrates the exemplary interconnect 14 in a substantially relaxed position. FIG. 14B illustrates the exemplary interconnect 14 in an at least partially compressed position. FIG. 14C illustrates the exemplary interconnect 14 in an at least partially expanded position.

FIGS. 15A to 15C illustrate another exemplary interconnect 14 having curved sections 56, 66 and angled transitions 60, 70 defining a first flexible lobe, a second flexible lobe and a third flexible lobe axially along the interconnect 14 in an alternative configuration to the lobes illustrated in FIGS. 14A to 14C. FIG. 15A illustrates the exemplary interconnect 14 in a substantially relaxed position. FIG. 15B illustrates the exemplary interconnect 14 in an at least partially compressed position. FIG. 15C illustrates the exemplary interconnect 14 in an at least partially expanded position.

Stents 10 in accordance with the present inventions may be manufactured using a wide variety of techniques that will be recognized by those skilled in the art upon review of the present disclosure. One exemplary method can include providing a segment of cylindrical walled material from which the stent 10 will be made. Depending upon the type of stent 10 to be made, any of the materials herein discussed or other materials that are well known in the art may be used depending upon the particular characteristics desired. The stent 10 is prepared by removal of material from the cylindrical wall, which material will not be part of the stent 10 to be formed. This may occur by mechanically cutting away material. Preferably, however, the cutting or material removal is automated. A computer aided laser-cutting device is one option. A computer aided water-jet cutting device is another option. In each case, software that guides the cutting tool will assure that only the material, which is intended to be removed, is in fact removed. Another removal technique is chemical etching of the cylinder wall. The portion of the cylinder to be retained as a part of the stent is protected from exposure to the chemical etching process. For example, in the case of a metallic stent, an etching agent might be one of a number of acids, which are well known in the art. A chemically protective agent, for example, a hydrophobic coating, such as a wax, may be applied over the entire exterior surface of the cylinder. Next, the protective coating is removed mechanically using a computer aided water jet cutting device, or the like, where etching is desired. If greater surface thickness is desired, wider areas need to be protected. If thinner surface thickness is desired, then narrower areas are protected. Alternatively, other means of selectively applying protective coatings, for example, photographically based methods, which are well known in the etching arts, may be used. Finally, the partially protected cylinder is immersed in an acid bath. Etching occurs throughout the interior cylinder surface but only at selected portions of the exterior. When the etching has proceeded to the extent that the etching from the exterior and interior surface has fully removed appropriate portions of the cylinder, the piece is removed from the acid. Next, the protective coating is removed. If the coating is wax, the wax may be removed by heating or by a wax solvent, which does not further affect the metal. Chemical etching is a suitable production method for low volume production. Higher volume production is believed to be more suitably achieved through the use of computer aided laser etching. The availability of using wider or narrower surface thickness, as well as different tubing wall thickness is considered an important means of obtaining stiffness or easier deformability in the desired devices of the present invention. Generally, thin wall tubing is believed to be preferable, but not absolutely required.

An alternate material from which expandable stents 10 in accordance with the present invention may be prepared is, without limit, stainless steel, particularly type 316 stainless steel, more preferably type 316 L or 316 Lvm stainless steel, but gold, platinum, e tantalum, silver and the like are also believed to be suitable. Other materials may include various polymers, composite materials and other materials as will be recognized by those skilled in the art upon review of the present disclosure. Some features for which the material may be selected are deformability and the ability to hold the shape once deformed. It may also desirable that the stent 10 be made from radiopaque materials or include radiopaque coatings over at least a portion of the stent 10. Stents 10 made of stainless steel which have a thickness of 0.005 inch are typically radiopaque, however, stents having lesser thicknesses, such as stents made specifically for use in coronary arteries which often requires thicknesses less than 0.005 inch (often for example, about 0.003 inch) may need to be coated with a radiopaque material such as 24 carat gold to a thickness of about 0.0002 inch. In addition, other coatings including specific functional agents may also be employed to address issues such as blood clotting (e.g. heparin and the like) or reduction in the amount of intimal hyperplasia and resulting restenosis (e.g. cytotoxic drugs, gene therapy agents and the like). Methods to coat metal prostheses to make them radiopaque or to minimize the risks due to blood clotting are well known in the art and any of these methods and the devices resulting from the use of these methods are all envisioned within the scope of the present invention.

It is understood that even though numerous characteristics and advantages of various embodiments of the present invention have been set forth in the foregoing description, together with details of the structure and function of various embodiments of the invention, this disclosure is illustrative only and changes may be made in detail, especially in matters of shape, size and arrangement of parts, within the principles of the present invention, to the full extent indicated by the broad general meaning of the terms in which the appended claims are expressed.

What is claimed is:

1. A stent comprising:
a proximal radially expandable segment;
a distal radially expandable segment;
a plurality of interconnects having a first arm and a second arm;
the plurality of interconnects secured to the proximal radially expandable segment; and,
the plurality of interconnects secured to the distal radially expandable segment.

2. The stent of claim 1, wherein the first arm and a second arm are symmetric in a curvilinear plane defined by an outer surface of the proximal and distal radially expandable segments.

3. An interconnect for securing a distal radially expandable segment to a proximal radially expandable segment comprising:
a first arm;
a second arm;
a proximal connector;
a distal connector; and,
the first arm and the second arm secured between the proximal connecter and the distal connector, the proximal connecter secured to a proximal radially expandable segment, and the distal connector secured to a distal radially expandable segment.

4. The interconnect of claim 3, wherein the first arm defines one or more curves extending from a central axis in a curvilinear plane defined by the outer surface of the radially expandable segments, and the second arm is substantially symmetrical to the first arm about the central axis in the curvilinear plane defined by the outer surface of the radially expandable segments.

5. The interconnect of claim 3, wherein the first arm defines one or more linear sections extending from a central axis in a curvilinear plane defined by the outer surface of the radially expandable segments, and the second arm is substantially symmetrical to the first arm about the central axis in the curvilinear plane defined by the outer surface of the radially expandable segments.
